(19) United States
(12) Patent Application Publication
(10) Pub. No.: US 2014/0276622 A1
(43) Pub. Date: Sep. 18, 2014

(54) NERVE BLOCK SYSTEM WITH CURVED INTRODUCER AND METHODS OF USE

(71) Applicant: N. Sandor Racz, Coppell, TX (US)

(72) Inventor: N. Sandor Racz, Coppell, TX (US)

(73) Assignee: Custom Medical Applications, Farmers Branch, TX (US)

(21) Appl. No.: 13/839,451

(22) Filed: Mar. 15, 2013

(51) Int. Cl. A61B 17/34; A61M 19/00

(52) U.S. Cl. CPC A61B 17/3401; A61M 19/00
USPC 604/512; 604/170.03

(57) ABSTRACT

Disclosed herein is a nerve block system comprising a nerve block needle and a curved introducer. Also disclosed are components of and methods of use of the nerve block system. The curved introducer of the nerve block system provides navigability and maintains column stiffness.

### FIELD

The disclosure relates generally systems and methods to control pain or perform diagnostics. In particular the disclosure relates to nerve block needles with curved introducers, related systems, and methods of use.

### BACKGROUND

Nerve block systems are used to inject anesthetic onto or near nerves for temporary control of pain or as a diagnostic tool. Nerve block needles are used to intentionally target specific areas and neural structures for injection. Sharp needles are predominantly used for quick and easy injections. Blunt needles where introduced to safely approach nerve structures without piercing or damaging the nerve. For the blunt needle to be placed, an introducer needle (shorter than the length of the blunt needle) is needed to penetrate the skin/muscle or ligament and then reach the target with the non-cutting blunt tip. An introducer is a tube through which another instrument, such as a needle, cannula, or catheter may be passed.

Curving radiofrequency needles aid in the accurate placement of the needle tip (“Bent tip electrical surgical probe” U.S. Pat. No. 6,146,380, incorporated by reference). The concept of curved needles was adopted for various other standard (non-radiofrequency) nerve block needles.

Doctors generally prefer to use smaller gauge needles to prevent excess bleeding and tissue damage where possible. However, whenever a small gauge nerve block needle is bent, the needle loses column stiffness making the needle harder to navigate and more prone to collapse. One nerve block needle bend may be a gentle sweep 1 cm back from the distal needle tip at a 15 degree angle. If angle is reduced or eliminated, the body of nerve block needle retains more stiffness but the nerve block needle becomes less directional.

It would therefore be advantageous to design a nerve block system capable of maintaining column stiffness and navigability.

### SUMMARY

An embodiment comprises the nerve block needle system comprises a curved introducer needle defined by a first inside diameter, a first outside diameter, and a first length, wherein the curved introducer needle is at least partially hollow; a nerve block needle defined by a second inside diameter, a second outside diameter, and a second length; and wherein the outside diameter of the nerve block needle is less than the inside diameter of the curved introducer needle. In another embodiment, the curved introducer needle is comprised of medical grade metal. In yet another embodiment, the curved introducer needle is comprised of hard plastic.

In an embodiment, the length of the curved introducer needle is less than the length of the nerve block needle. In still another embodiment, the angle of the curved introducer needle is 45 degrees or less. In yet another embodiment, the angle of the curved introducer needle is 15 degrees.

In an embodiment, the curved introducer needle is sharp. In another the curved introducer needle comprises a bevel tip. In yet another embodiment, the bevel tip is cutting. In still another embodiment, the bevel tip is piercing. In still another embodiment, the nerve block needle is comprised of medical grade metal.

In an embodiment, the nerve block needle is comprised of hard plastic. In another embodiment, the nerve block needle is curved. In yet another embodiment, the outer diameter of the nerve block needle is 22 gauge or less. In still another embodiment, the angle of the nerve block needle is 15 degrees or less. In yet another embodiment, the angle of the nerve block needle is 5 degrees. In still another embodiment, the angle of the nerve block needle is 0 degrees.

In an embodiment, the nerve block needle comprises a blunt tip. In another embodiment, the nerve block needle comprises a retrofitted occluded sharp tip with side port injection. In still another embodiment, the nerve block needle comprises a sharp bevel tip.

Another embodiment comprises a method of using the nerve block system comprising the steps of: locating a site for injection on a patient; inserting into the patient at least a portion of a nerve block system comprising a curved introducer needle defined by a first inside diameter, a first outside diameter, and a first length, wherein the curved introducer needle is at least partially hollow; a nerve block needle defined by a second inside diameter, a second outside diameter, and a second length; wherein the outside diameter of the nerve block needle is less than the inside diameter of the curved introducer needle; and wherein the curved introducer needle is used for access across a tissue of a patient; wherein the nerve block needle is inserted through the inside diameter of the curved introducer needle; and injecting the medicament into the patient.

In another embodiment, the curved introducer has a sharp end. In yet another embodiment, the nerve block needle has a blunt end. In still another embodiment, the nerve block needle is curved.

In another embodiment, the outer diameter of the nerve block needle is 22 gauge or less. In still another embodiment, the angle of the nerve block needle is 15 degrees or less. In yet another embodiment, the angle of the nerve block needle is 5 degrees. In another embodiment, the angle of the nerve block needle is 0 degrees.

The foregoing has outlined rather broadly the features of the present disclosure in order that the detailed description that follows may be better understood. Additional features and advantages of the disclosure will be described hereinafter, which form the subject of the claims.

### BRIEF DESCRIPTION OF THE DRAWINGS

In order that the manner in which the above-recited and other enhancements and objects of the disclosure are obtained, a more particular description of the disclosure briefly described above will be rendered by reference to specific embodiments thereof which are illustrated in the appended drawings. Understanding that these drawings depict only typical embodiments of the disclosure and are therefore not to be considered limiting of its scope, the disclosure will be described with additional specificity and detail through the use of the accompanying drawings in which:

FIG. 1 is an illustration of left, a 15 degree curved blunt nerve block needle with a straight sharp introducer needle and right, a 5 degree curved blunt nerve block needle with a 15 degree curved sharp introducer needle;

FIG. 2 is an illustration of left, a 15 degree curved nerve block blunt needle within a straight sharp introducer needle and right, a 5 degree curved blunt nerve block needle within a 15 degree curved sharp introducer needle;

FIG. 3 is an illustration of a curved sharp introducer needle; and

FIG. 4 is an illustration of a curved blunt nerve block needle within a curved sharp introducer needle.

FIG. 5 is a cross-sectional illustration of a curved sharp introducer needle; and

FIG. 6 is a cross-sectional illustration of a curved blunt nerve block needle within a curved sharp introducer needle.

### DETAILED DESCRIPTION

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings making apparent to those skilled in the art how the several forms of the disclosure may be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary 3^{rd} Edition.

As used herein the term, “health professional” means and refers to an individual that is qualified to identify, prevent, or treat a medical condition in a patient.

As used herein the term, “patient” means and refers to one in need of a treatment.

As used herein, a “fluid” is a continuous, amorphous substance whose molecules move freely past one another and that has the tendency to assume the shape of its container, for example, a liquid or a gas.

As used herein, the term “medicament(s)” means and refers to all types of substances (e.g., including solutions and suspensions) that have a beneficial, desired or therapeutic effect. Non-limiting examples of medicaments suitable for use in the disclosure methods include anesthesia, biologically active agents, such as small molecule drugs, proteinaceous substances, polynucleotides or nucleic acids (e.g. heterologous DNA, or RNA) and vectors, liposomes, and the like, containing such nucleic acids or polynucleotides, as well as liquid preparations or formulations thereof.

As used herein, the term “medical instrument” means and refers to any item, instrument or structure capable of connecting to a catheter, such as, but not limited to a stimulation device, tubing, piping, a medicament delivery system, a meter, a liquid repository (such as an I.V. bag), a syringe, and/or the like.

As used herein, the term “medical grade metals” means and refers to any metal or alloy thereof that is capable of being used in a medical application, including, but not limited to, injection, implantation, and instrumentation. Examples of medical grade metals include, but are not limited to, stainless steels, cobalt-chromium based alloys, and titanium alloys.

As used herein, the term “plastic” means and refers to any synthetic material made from an organic polymer. Examples of medical plastics include, but are not limited to, polyethylene, polypropylene, polystyrene, polyvinyl chloride, acrylonitrile butadiene styrene (ABS), polycarbonate, polyurethanes, polyamides, polyetheretherketone (PEEK), polysulfones, and elastomers, and alloys thereof.

As used herein, the term “attached,” or any conjugation thereof describes and refers to the at least partial connection of two items.

A typical needle includes four integral elements: a proximal “hub” or connector for fluidic attachment to a medical instrument; a straight tubular “shaft” which is typically at least partially hollow; a conical or tapered portion at the distal end of the straight tubular shaft portion; and a distal end or “tip”.

More particularly, embodiments of the present disclosure generally comprise a surgical instrument comprising an elongated at least partially hollow shaft, generally extending from a proximal end to a distal end; a sharp needle at least partially occluded, closed distal end or tip end; a connector end; and, at least one side port located coaxially along the shaft. In further embodiments, the surgical instrument further comprises a medical instrument, i.e., any item, instrument or structure capable of connecting to a connector, such as, but not limited to a stimulation device, tubing, piping, a medicament delivery system, a meter, a liquid repository (such as an I.V. bag), a syringe, and/or the like connected to the connector end.

An embodiment of the disclosure includes a nerve block system capable of maintaining column stiffness and navigability. Insertion through a curved introducer needle allows the curved nerve block needle and entire nerve block system to be maneuverable while not compromising column stiffness.

Various embodiments of the disclosure further comprise methods of use. In an exemplary, non-limiting embodiment of a method of the present disclosure wherein a patient is in need of treatment by a medicament comprising the steps of: locating a site for injection on a patient; inserting into the patient at least a portion of a nerve block system comprising an at least partially hollow curved introducer needle being defined by a first inside diameter, a first outside diameter, and a first length, wherein said curved introducer needle has a sharp end; a nerve block needle being defined by a second inside diameter, a second outside diameter, and a second length, wherein the nerve block needle has a blunt end; wherein the outside diameter of the nerve block needle is less than the inside diameter of the curved introducer needle; and wherein the curved introducer needle is used for access across a tissue of a patient; wherein the nerve block needle is inserted through the inside diameter of the curved introducer needle; and injecting the medicament into the patient.

Further embodiments comprise stimulating the site and/or ablating the site.

In further embodiments, the patient is in need of access to a particular tissue.

In various other embodiments, the disclosure's blunt nerve block needle and/or blunt surgical assemblage is ideally suited for injection into tissue of medicaments containing nucleic acid encoding a therapeutic agent (or cells containing such nucleic acid), For example, the nerve block needle (when attached to an appropriate needle or catheter) or disclosure surgical assemblage can be used to inject medicament(s) into the wall of a beating heart or other internal organ, without substantial loss of the medicament at the surface of the body wall and without substantial damage to tissue at the injection site caused by injectate.

As used herein, the term “normal insertion procedure” means and refers to a typical surgical and/or insertion procedure as disclosed in Heavner et al., Sharp Versus Blunt Needle: A Comparative Study of Penetration of Internal Structures and Bleeding in Dogs, World Institute of Pain, Pain Practice, 3:3, 226-231 (2003).

Various embodiments of the disclosure methods and devices are designed for injection of minute amounts of fluid medicaments into tissue or a body wall, for example, an interior body wall. The therapeutic amount of the medicament to be administered according to the disclosure method will vary depending upon the therapeutic goal to be accomplished, the size and age of the subject, the pharmacokinetics of the injectate, and the like. However, a therapeutic amount according to the present disclosure is typically in the range from about 0.1 cc to about 5.0 cc.

Smaller gauge needles are beneficial to minimize excess bleeding and tissue damage. Bending small gauge nerve block needles causes a loss of column stiffness. This loss makes the needle harder to navigate and more prone to collapse.

The nerve block needle bend may be a gentle sweep 1 cm back from the distal needle tip at a 15 degree angle. If the 15 degree angle is reduced or eliminated, the body of the nerve block needle retains more stiffness but the needle becomes less directional. By including a bend to the rigid introducer needle, the system is easier to navigate. The inner nerve block needle can be made smaller, with a reduced number of degrees of the bend angle or no bend at all to maintain column stiffness. This decreases the possibility of needle collapse. The nerve block needle with curved introducer makes it easier to navigate a safer and smaller nerve block needle, resulting in less tissue trauma due to more accurate placement. A curved introducer needle provides less tissue trauma and more accurate placement.

The bend of the introducer needle provides the ability to place the nerve block needle along a nerve without the loss of column strength that would come from a similar bend in the nerve block needle itself.

The nerve block system may be attached to a syringe or other mechanism of fluid delivery. In an embodiment, the nerve block needle may be attached to a syringe. In further embodiments, a connector may be present to aid in attachment of the nerve block needle to a syringe.

### Introducer Needle

The introducer needle may vary in overall length but the introducer needle is shorter than the accompanying nerve block needle. In one embodiment, the introducer needle is 1.25 inches.

The gauge size of the introducer may vary but needs to have a large enough inside diameter to pass the accompanying inner nerve block needle. In one embodiment, the gauge is 20 gauge.

The introducer may be comprised of various medical grade metals or various hard plastics.

Various medical grade metals include, but are not limited to, stainless steels, cobalt-chromium based alloys, and titanium alloys.

Various hard plastics may include, but are not limited to, polyethylene, polypropylene, polystyrene, polyvinyl chloride, acrylonitrile butadiene styrene (ABS), polycarbonate, polyurethanes, polyamides, polyetheretherketone (PEEK), polysulfones, and elastomers, and alloys thereof.

The angle of the bend of the introducer may be 45 degrees or less. In one embodiment the angle of the bend is 15 degrees.

The tip of the introducer needle may be blunt or sharp. A benefit of a blunt tipped introducer is that damage to anatomical structures due to the tip of the instrument is minimized. A benefit of sharp tipped introducer is that the tip is able to pierce the skin and other structures of the patient in order to provide treatment at the anatomical structure. A sharp tipped introducer needle may comprise a piercing or cutting bevel tip.

### Nerve Block Needle

The nerve block needle may vary in overall length but the needle is longer in length than the accompanying introducer. In one embodiment, the nerve block needle is 3.5 inches.

The outer gauge size of the nerve block needle may be 22 gauge or smaller (in diameter). The needle must have a smaller outer diameter measurement than the inner diameter of the accompanying outer introducer needle. In one embodiment, the gauge is 25. A 22 gauge needle has an inner diameter of 1/25 inches while a 22 gauge needle has a diameter of 1/22 inches.

The nerve block needle may be comprised of various medical grade metals or various hard plastics.

Various medical grade metals include, but are not limited to, stainless steels, cobalt-chromium based alloys, and titanium alloys.

Various hard plastics may include, but are not limited to, polyethylene, polypropylene, polystyrene, polyvinyl chloride, acrylonitrile butadiene styrene (ABS), polycarbonate, polyurethanes, polyamides, polyetheretherketone (PEEK), polysulfones, and elastomers, and alloys thereof.

The angle of the bend of the nerve block needle may range from less than 15 degrees to no bend at all. In one embodiment the angle of the bend is 5 degrees. In another embodiment, the angle of the bend is 15 degrees.

The tip of the nerve block needle may be blunt or sharp. Needle tips of the nerve block needle may include, but are not limited to blunt tip, retrofitted occluded sharp tip with side port injection, or standard sharp bevel tip. A benefit of a blunt tipped needle is that damage to anatomical structures due to the tip of the instrument is minimized. A benefit of sharp tipped nerve block needle is that the tip is able to pierce the skin and other structures of the patient in order to provide treatment at the anatomical structure.

As shown in FIG. 1, Left, is a nerve block system is comprised of a nerve block needle 100 and a straight introducer needle 120. Depicted is a 25 gauge 3.5 inch in length 15 degree curved blunt nerve block needle 100 that may be used with a 20 gauge 1.25 inch in length straight sharp introducer needle 120. The nerve block needle has a blunt end 112 and the introducer needle has a sharp end 122. Also depicted in FIG. 1 is a curve 114 in the nerve block needle.

As shown in FIG. 1, Right, is a nerve block system comprised of a nerve block needle 140 and a straight introducer 160. Depicted is a 25 gauge 3.5 inch in length 5 degree curved blunt nerve block needle 140 with a 20 gauge 1.25 inch in length 15 degree curved sharp introducer needle 160. The nerve block needle has a blunt end 142 and the introducer needle has a sharp end 162. Also depicted in FIG. 1 is a curve 144 in the nerve block needle 140 and curve 164 in the introducer needle 160.

As shown in FIG. 2, Left, is an 25 gauge 3.5 inch in length 15 degree curved blunt nerve block needle 200 may be placed within a 20 gauge 1.25 inch in length straight sharp introducer needle 220. Depicted in FIG. 2 is a nerve block system comprised of a nerve block needle 200 and a straight introducer 220. The nerve block needle has a blunt end 212 and the introducer needle has a sharp end 222. Also depicted in FIG. 2 is a curve 214 in the nerve block needle 200.

As shown in FIG. 2, Right, an embodiment of the disclosure is a 25 gauge 3.5 inch in length 5 degree curved blunt nerve block needle 240 within a 20 gauge 1.25 inch in length 15 degree curved sharp introducer needle 260. Depicted in FIG. 2 is a nerve block system comprised of a nerve block needle 240 and a straight introducer 260. The nerve block needle has a blunt end 242 and the introducer needle has a sharp end 262. Also depicted in FIG. 2 is a curve 244 in the nerve block needle 240 and curve 264 in the introducer needle 260.

As shown in FIG. 3, an embodiment of the disclosure is a curved sharp introducer needle 300. The introducer needle has a sharp end 322. The angle 310 of the bend of the introducer needle 300 is measured by comparison of the curved portion and straight portion of the shaft 320 of the introducer needle.

As shown in FIG. 4, a nerve block needle system 400 is depicted comprising a curved blunt nerve block needle 420 within a curved sharp introducer needle 440. The angle 410 of the bend of the introducer needle 440 is measured by comparison of the curved portion and straight portion of the shaft 430 of the introducer needle 440. The angle 450 of the bend of the nerve block needle 420 is measured by comparison of the curved portion and straight portion of the shaft 460 of the nerve block needle 420. Also depicted is connector 470.

As shown in FIG. 5, a cross-sectional view of a curved sharp introducer needle 300. The introducer needle has a sharp end 322. The angle 310 of the bend of the introducer needle 300 is measured by comparison of the curved portion and straight portion of the shaft 320 of the introducer needle.

As shown in FIG. 6, a cross-sectional view of a nerve block needle system 400 is depicted comprising a curved blunt nerve block needle 420 within a curved sharp introducer needle 440. The angle 410 of the bend of the introducer needle 440 is measured by comparison of the curved portion and straight portion of the shaft 430 of the introducer needle 440. The angle 450 of the bend of the nerve block needle 420 is measured by comparison of the curved portion and straight portion of the shaft 460 of the nerve block needle 420. Also depicted is connector 470.

Increased user safety during needle use or operation is a benefit in a needle with an at least partially occluded tip that was still able to engage and/or enter at least one tissue of a patient. Further, it is known that needles with a blocked tip (distal end) and with a side opening are used for procedures such as thoracentesis which involves inserting the needle through the thoracic cage into the pleural space between the lung and the chest wall to draw off fluid for diagnostic or therapeutic purposes. Thoracentesis needles generally consist of an orifice free, sharp conical end and a circular side hole for draining fluid.

A nerve block is a chemical paralysis of the nerve by a local anesthetic agent injected in the vicinity of the nerve. In one embodiment, the procedure is a stellate ganglion nerve block. When a predetermined amount of blocking agent is correctly injected, the middle cervical ganglion, the intermediate ganglion, the stellate ganglion and the second, third, and fourth thoracic ganglia are anesthetized (“blocked”). Additionally, the superior cervical ganglion is also anesthetized because the nerve fibers that form this ganglion extend through the above-mentioned ganglia. Thus, when a stellate ganglion sympathetic block is correctly executed, the entire cervicothoracic portion of the sympathetic nervous system is blocked. Generally, for short time duration blocks, local anesthetic drugs such as novocaine, pontocaine, xylocalne, metycaime and intracaine, nupercaine and the like are used. For long time duration blocks, alcohol and phenol are generally used.

In various embodiments of procedures for blocks, a physician relies on superficial landmarks (i.e. bones, cartilage, muscles, tendons, and blood vessels which are near the skin surface) to locate the area of insertion of the needle. Deep landmarks (i.e. bones, periosteum, fascial planes, tendons, and blood vessels) that cannot be seen or palpated can only be felt with the tip or distal end of the needle. Thus, the accuracy in identifying these deep landmarks depends greatly on the educated touch of the physician.

Exemplary, non-limiting embodiments of medical instrument, surgical instruments, and/or the like that can be modified according to various teachings of the present disclosure include, but are not limited to, U.S. Pat. Nos. 6,949,087; 6,855,132; 6,558,353; 6,547,769; 6,387,163; 6,245,044; 5,871,470; 5,865,806; 5,836,914; 5,817,074; 5,800,445; 5,730,749; 5,669,882; 5,628,734; 5,573,519; 5,571,091; 5,480,389; 5,466,225; 5,336,191; 5,312,360; 5,304,141; 5,250,035; 5,242,410; 5,106,376; 4,994,034; 4,973,313; 4,629,450; 4,317,445; 4,308,875; 4,230,123; 3,856,009; 3,565,074 and, 2,922,420, the contents of which are hereby incorporated by reference as if they were presented herein in their entirety. In general, any catheter may be used with the various embodiments of the present disclosure.

A tip end or distal end of a needle may comprise a shoulder along the juncture of the tip and the shaft; at least one generally beveled edge extending from the shoulder to the point of the tip. In an embodiment, a beveled edge extends circumferentially from about the shoulder to the point whereby the point of the tip is still capable of being inserted into at least one tissue of a patient, but the exposed edge or point is not as sharp as a needle point. In an alternate embodiment, the point of the tip is defined by a beveled edge extending from the shoulder to the point in a quadrangular arrangement, such that the beveled edges meet in a stealth arrangement whereby the point of the tip is still capable of being inserted into a tissue of a patient, but the exposed edge or point is not as sharp as a needle point. In an alternate embodiment, the point of the tip is defined by a beveled edge extending from the shoulder to the point in a triangular arrangement whereby the point of the tip is still capable of being inserted into a tissue of a patient, but the exposed edge or point is not as sharp as a needle point. In an alternate embodiment, the point of the tip is defined by a beveled edge extending from the shoulder to the point to form a blade whereby the point of the tip is still capable of being inserted into a tissue of a patient, but the exposed edge or point is not as sharp as a needle point. In an alternate embodiment, the point of the tip is defined by a beveled edge extending from the shoulder to the point whereby the point of the tip is still capable of being inserted into a tissue of a patient, but the exposed edge or point is not as sharp as a needle point. In an embodiment, a point associated with a tip of a needle of the present disclosure may be defined by any number of bevels or facets whereby the point of the tip is still capable of being inserted into a tissue of a patient, but the exposed edge or point is not as sharp as a needle point.

Angles of bevel for various embodiments can vary. In general, an angle of bevel can be from about 1° to about 90°. In an embodiment, by way of example, the angle of bevel is between about 15° and about 65°. In an alternate embodiment, again by way of example, the angle of bevel is about 45°. The angle of bevel is capable of being the same for all facets. However, in various embodiments, the angle of bevel varies for at least one facet as compared to the remaining facet(s). Design characteristics to consider when determining a proper bevel include, but are not limited to, the tissue to be punctured, the delicate organs about the incision, the medicament to be administered, and/or the like. In an embodiment, an angle of 45° yields a tip length of about 0.05055 mm for a 20 gauge needle and approximately 0.03995 mm for a 22 gauge needle. However, tip lengths may vary according to the particular embodiment.

A side port of the present disclosure is generally a port extending from the exterior of the needle to the interior of the needle. The shape of a port can vary. In an embodiment, a port is circular. In an alternate embodiment, a port is ovular. In an alternate embodiment, a port is a quadrangular port, such as a rectangle or a square. In an alternate embodiment, the port is triangular. In general, ports of the present disclosure can be any shape sufficient to permit fluid aspiration therethrough. A side port of the present disclosure may be further characterized by the associated edge of the port on the shaft. In an embodiment, a port has a slightly inwardly beveled edge extending from the exterior surface of the shaft to the interior surface. In an alternate embodiment, a port has a slightly outwardly beveled edge extending from the interior surface of the shaft to the exterior surface. In an embodiment, the degree of bevel can be used to change the pressure of the medicament as it enters the target tissue; facilitate a change in the degree of spread of the medicament; and, allow for a smooth surface as the shaft is inserted to the target tissue. In various embodiments, a side port of the present disclosure can be located at any location about the needle. In an embodiment, the at least one side port is aligned with the point or edge of the distal portion of the needle. In an alternate embodiment the at least one side port is located 180-degrees from alignment with the point or edge of the distal portion of the needle.

In an embodiment, an occluded nerve block needle comprises a weeping tip portion for microinjection of medicaments into a tissue. In an embodiment, again by way of example, the surgical instrument comprises a nonporous hollow nerve block needle shaft having a connector end adapted to mate with a surgical instrument, a porous distal portion in fluid-tight connection to the nerve block needle shaft, and a closed tip. The porous distal portion of the nerve block needle may be adapted to cause a liquid injectate to weep or ooze therefrom multidirectionally under injection pressure while the distal portion and point of the nerve block needle are inserted into a tissue. In an embodiment, the porous distal portion oozes at a substantially uniform rate.

A cannula or needle associated with various embodiments of a surgical instrument is generally a cylindrical structure extending from the connector to the tip end. In an embodiment, the shaft is of a generally constant circumference. The shaft is capable of being differentiated by an inside diameter and an outside diameter. In an embodiment, an outside diameter is between about 0.0355 to about 0.03600 mm and an inside diameter is between about 0.0230 to about 0.0245 mm. In an alternate embodiment, an outside diameter between about 0.0280 to 0.205 mm and an inside diameter between about 0.0155 to 0.0170 mm. Generally, an inside diameter and an outside diameter are capable of being any desired measurement.

Connector ends of the present disclosure comprise an attachment means for attaching the sharp nerve block needle at least partially occluded, closed distal end to the shaft. Further, connector ends of the present disclosure comprise an attachment means for attaching a medical instrument. The connection or connections at the connector end may be any type of connection common in the art, such as, for example, and not by way of limitation, a luer lock connector, a threaded attachment, an interference fit attachment, a clamp, a system utilizing a dowel, two or more of the aforesaid in combination, and/or the like.

Embodiments of connectors of the present disclosure optionally may comprise at least one wing. A wing of the present disclosure may be any structure. In an embodiment, the wing is an extension of the connector, such as, but not limited to, a rigid flap, a rigid bar, a flexible flap, a flexible bar, and/or the like. In further embodiments at least two wings extend from about the connector, such as, for example, and not by way of limitation, two wings extending evenly spaced circumferentially about the connector. It is common in the medical field that needles are inserted into patients for a variety of procedures. An issue that arises is how to secure or stabilize the needle before during and/or after insertion. A hemostat, pliers, and/or the like are often used to secure an area about the connector to secure it. Numerous procedures, such as, but not limited to, fluoroscopic guidance procedures, NMR procedures, X-ray procedures, direct viewing procedures, and/or the like would find a benefit in a winged structure.

In various embodiments, a measurement that is capable of differentiating various embodiments of the present disclosure is the measurement from the shoulder of the tip to an edge of a port. In general, embodiments of the present disclosure comprise any shaft wherein the nerve block needle is not adjacent to a shoulder of the tip end. By not being adjacent means that a shoulder of a tip and a port on the shaft are at least separated by some distance. In an embodiment, by way of example, the distance is between about 0.01 mm to about 100.0 mm. In an alternate embodiment, again by way of example, the distance is between about 0.5 mm to about 75.0 mm. In an alternate embodiment, again by way of example, the distance is between about 1.0 mm to about 50.0 mm. In an alternate embodiment, again by way of example, the distance is between about 5.0 mm to about 25 mm. In general, the distance between the shoulder of the tip and the port of the associated shaft can vary as needed for the particular application.

Accordingly, a further embodiment of the present disclosure comprises a surgical instrument comprising an elongated at least partially hollow shaft comprising a sharp, occluded nerve block needle means for insertion into a patient; a connector end connected through a connection to an instrument; and, at least one side port located coaxially along the shaft.

An exemplary embodiment of a method of the present disclosure comprises a method for administering a medicament to a patient comprising the steps of locating a site for injection; inserting at least a portion of a surgical instrument into the site, the surgical instrument comprising an elongated at least partially hollow shaft having a sharp needle at least partially occluded, closed distal end; a connector end; and, at least one side port located coaxially along the shaft; and, injecting the medicament into the patient. In an embodiment, the medicament is anesthesia.

Further embodiments of a method of the present disclosure comprise stimulating a tissue. Yet further embodiments of a method of the present disclosure comprise ablating at least a portion of the tissue about a site. Other embodiments comprise probing a tissue. And yet further embodiments of a method of the present disclosure comprise preparing a patient for administering a medicament.

In an embodiment of an administration of a block, a method of the present disclosure comprises placing a patient in a supine position or extended position, without a pillow, with the patient's head in a neutral position. While standing on side of the body that is to be blocked, the physician tactilely locates the cricoid cartilage. The needle is then inserted in a position approximately one finger breadth below the cricoid cartilage, between the carotid sheath and the trachea on the side to be blocked, while aiming slightly medially until bony contact is made with the ventral lateral side of the body of the seventh cervical vertebra. When the needle is in said position, the anesthesia is injected. An embodiment of the disclosure allows for directional injection out the side port onto the surface of the anterior longitudinal ligament. This allows the anesthesia to spread onto the surface of the longus coli muscle toward the stellate ganglion. Thus, an injection via the side port will achieve an effective stellate ganglion sympathetic block.

In another embodiment according to the present disclosure, there are provided methods for injecting a medicament into tissue in a subject in need thereof. The disclosure injection method comprises inserting the distal portion of the nerve block needle into the tissue of the subject and causing a therapeutic amount of the medicament to flow from the nerve block needle into the tissue without substantial leakage or loss of the medicament at the surface of the tissue.

The disclosure method using the nerve block needle (or surgical assemblage) with porous distal portion is designed for injection of minute amounts of fluid into tissue or a body wall, hence the use of the term “microinjection” herein. However other amounts may also be injected.

In another embodiment according to the present disclosure, there are provided methods for injecting a medicament into a subject in need thereof, comprising inserting the distal portion of the nerve block needle into an interior body wall or tissue of the subject and applying sufficient pressure to a liquid medicament in fluid communication with the distal portion of the nerve block needle to expel the medicament such that the medicament flows into the interior body wall or tissue without substantial leakage or loss of the medicament at the surface of the body wall.

In yet another embodiment, the present disclosure provides a method for injecting a medicament into tissue in a subject in need thereof comprising: inserting the distal portion of a curved introducer needle and a nerve block needle into the tissue of the subject and causing a therapeutic amount of the medicament to flow from the nerve block needle into the tissue without substantial damage to the tissue of the subject caused by injectate.

In one embodiment, the medical professional locates a site for injection on a patient; inserts into the patient at least a portion of a nerve block system comprising an at least partially hollow curved introducer needle being defined by a first inside diameter, a first outside diameter, and a first length, wherein said curved introducer needle has a sharp end; a nerve block needle being defined by a second inside diameter, a second outside diameter, and a second length, wherein the nerve block needle has a blunt end; wherein the outside diameter of the nerve block needle is less than the inside diameter of the curved introducer needle; and wherein the curved introducer needle is used for access across a tissue of a patient; wherein the nerve block needle is inserted through the inside diameter of the curved introducer needle; and injects the medicament into the patient.

### EXAMPLES

### Example 1

A health professional may locate a site for injection on a patient. At least a portion of a nerve block system may be inserted into the patient. The nerve block system may comprise a sharp ended hollow curved introducer needle and a blunt ended nerve block needle with an outside diameter that is smaller than the inside diameter of the curved introducer needle. The curved introducer needle may be used for access across a tissue of a patient. The open blunt distal end of the nerve block needle may be inserted within the introducer needle to minimize damage to internal anatomical structures. The medicament may then be injected into the patient.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A nerve block needle system comprising:
a curved introducer needle defined by a first inside diameter, a first outside diameter, and a first length, wherein the curved introducer needle is at least partially hollow;
a nerve block needle defined by a second inside diameter, a second outside diameter, and a second length; and
wherein the outside diameter of the nerve block needle is less than the inside diameter of the curved introducer needle.

2. The nerve block needle system of claim 1 wherein the curved introducer needle is comprised of medical grade metal or hard plastic.

3. The nerve block needle system of claim 1 wherein the length of the curved introducer needle is less than the length of the nerve block needle.

4. The nerve block needle system of claim 1 wherein the angle of the curved introducer needle is 45 degrees or less.

5. The nerve block needle system of claim 4 wherein the angle of the curved introducer needle is 15 degrees.

6. The nerve block needle system of claim 1 wherein the curved introducer needle comprises a sharp tip.

7. The nerve block needle system of claim 1 wherein the curved introducer needle comprises a bevel tip.

8. The nerve block needle system of claim 8 wherein the bevel tip is capable of cutting.

9. The nerve block needle system of claim 8 wherein the bevel tip is capable of piercing.

10. The nerve block needle system of claim 1 wherein the nerve block needle is comprised of medical grade metal or hard plastic.

11. The nerve block needle system of claim 1 wherein the nerve block needle is curved.

12. The nerve block needle system of claim 1 wherein the outer diameter of the nerve block needle is 22 gauge or less.

13. The nerve block needle system of claim 1 wherein the angle of the nerve block needle is 15 degrees or less.

14. The nerve block needle system of claim 13 wherein the angle of the nerve block needle is 5 degrees.

15. The nerve block needle system of claim 13 wherein the angle of the nerve block needle is about 0 degrees.

16. The nerve block needle system of claim 1 wherein the nerve block needle comprises a blunt tip or a sharp bevel tip.

17. The nerve block needle system of claim 1 wherein the nerve block needle comprises a retrofitted occluded sharp tip with side port injection.

18. A method of using the nerve block system comprising the steps of:
locating a site for injection on a patient;
inserting into the patient at least a portion of a nerve block system comprising
a curved introducer needle defined by a first inside diameter, a first outside diameter, and a first length, wherein the curved introducer needle is at least partially hollow;
a nerve block needle defined by a second inside diameter, a second outside diameter, and a second length;
wherein the outside diameter of the nerve block needle is less than the inside diameter of the curved introducer needle; and
wherein the curved introducer needle is used for access across a tissue of a patient;
wherein the nerve block needle is inserted through the inside diameter of the curved introducer needle; and
injecting the medicament into the patient.

19. The method of claim 21 wherein the outer diameter of the nerve block needle is 22 gauge or less.

20. The method of claim 21 wherein the angle of the nerve block needle is 15 degrees or less.
